(19) United States
(12) Patent Application Publication
(10) Pub. No.: US 2025/0360254 A1
(43) Pub. Date: Nov. 27, 2025

(54) INTRAVENOUS GAS EXCHANGE SYSTEM

(71) Applicant: Regents of the University of Minnesota, Minneapolis, MN (US)

(72) Inventors: Demetris Yannopoulos, Edina, MN (US); Frank S. Bates, Saint Louis Park, MN (US); Marc Andrew Hillmyer, Minneapolis, MN (US)

(21) Appl. No.: 19/107,459

(22) PCT Filed: Sep. 1, 2023
(86) PCT No.: PCT/US2023/073342

§ 371 Date: Feb. 27, 2025

Related U.S. Application Data
(60) Provisional application No. 63/374,444, filed on Sep. 2, 2022.

(51) Int. Cl. A61M 1/16

(52) U.S. Cl. CPC A61M 1/1698

(57) ABSTRACT

A medical device includes an elongated member configured to be inserted into a vessel of a patient. The elongated member includes a permeable membrane that defines a wall of the elongated member and a flow channel within the wall. The permeable membrane defines nanopores extending from an exterior surface of the permeable membrane to tire flow channel. Idle nanopores are configured to enable diffusion of a pressurized gas from out of the flow channel and. into a fluid within the vessel. The nanopores are configured to form gas nanobubbles at the exterior surface of the permeable membrane as the pressurized gas diffuses out from the flow channel and into the fluid within the vessel.

This application is a PCT application that claims priority to and the benefit of U.S. Provisional Patent Application No. 63/374,444, filed Sep. 2, 2022, the entire content of which is incorporated herein by reference.

### TECHNICAL FIELD

The invention relates to medical devices, and more specifically, devices that provide intravenous gas exchange.

### BACKGROUND

The human body depends on intact lung function and appropriate ventilation/perfusion (V/Q) matching to exchange gasses and receive oxygen. Whenever the lung function or V/Q matching fails, the ability of the body to adjust is limited and leads to acute shock, blood hypoxemia, and tissue hypoxia that if not reversed swiftly leads to organ damage or death. A large number of medical conditions can lead to a decrease in oxygen delivery, with failure of the cardiorespiratory system and necessity of intubation to counteract hypoxemia.

### SUMMARY

This disclosure describes systems, devices, and techniques configured to intravenously exchange gasses, such as to deliver a large volume of oxygen. For example, a system may include a medical device configured to exchange gasses, such as oxygen and carbon dioxide, through a surface (e.g., a permeable membrane) of the medical device and based on the needs of the patient. The system may be configured to control the exchange of gasses via the medical device by controlling, for example, the pressure and flow rate of the gas flowing through the medical device. In examples, the system controls the flow rate of the gas by managing the resistance (e.g., by opening or closing a valve) to the return flow of the gas.

In some examples, a medical device comprises: an elongated member configured to be inserted into a vessel (e.g., a vein, an artery, etc.) of a patient, wherein the elongated member comprises a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

In some examples, a method comprises: inserting a medical device into a vessel of a patient, wherein the medical device comprises an elongated member comprising a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel; and delivering the pressurized gas through the flow channel of the permeable membrane.

In some examples, a system comprises: a container defining a cavity configured to contain a pressurized gas; and a medical device, fluidically coupled to the container, comprising: an elongated member configured to be inserted into a vessel of a patient, wherein the elongated member comprises a permeable membrane that defines a wall of the elongated member and a flow channel within the wall, wherein the permeable membrane defines nanopores extending from an exterior surface of the permeable membrane to the flow channel, wherein the nanopores are configured to enable diffusion of the pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

In some examples, a system comprises: a container defining a cavity configured to contain a pressurized gas; a pressurization device configured to pressurize gas; and a medical device, fluidically coupled to the container, comprising: an elongated member configured to be inserted into a vessel of a patient, wherein the elongated member comprises a permeable membrane that defines a wall of the elongated member and a flow channel within the wall, wherein the permeable membrane defines nanopores extending from an exterior surface of the permeable membrane to the flow channel, wherein the nanopores are configured to enable diffusion of the pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a conceptual diagram of an example system configured to intravenously deliver a gas to a patient, in accordance with one or more aspects of this disclosure.

FIG. 2 is a conceptual diagram of another example system configured to intravenously deliver a gas to a patient, in accordance with one or more aspects of this disclosure.

FIG. 3 is a conceptual diagram of an example medical device, in accordance with one or more aspects of this disclosure.

FIG. 4 is a conceptual diagram of another example medical device, in accordance with one or more aspects of this disclosure.

FIG. 5 is a conceptual diagram of yet another example medical device, in accordance with one or more aspects of this disclosure.

FIG. 6 is a flowchart of an example technique of an example system, in accordance with one or more techniques of this disclosure.

FIG. 7 is a flowchart of an example technique of a closed loop control system of the system, in accordance with one or more techniques of this disclosure.

FIG. 8 is a flowchart of an example technique of an example system. in accordance with one or more techniques of this disclosure.

### DETAILED DESCRIPTION

A medical device may be positioned within a vessel (e.g., a vein, an artery, etc.) of a patient and configured to deliver gases to and/or remove gases from the patient. In some cases, the medical device may deliver gas (e.g., oxygen, nitrous oxide, etc.) to patients at substantially atmospheric pressure (e.g., about 1 atmospheric pressure (atm)). However, the amount of gas (e.g., the molecular quantity) that may be diffused from the medical device at atmospheric pressure may be so small that metabolic requirements (e.g., oxygen requirements) and/or other requirements of the patient are not met. For example, delivery of gas at atmospheric pressure with a conventional medical device may be insufficient to meaningfully treat a patient with oxygen deficiency. Thus, delivery of oxygen at atmospheric pressure may not be a viable solution, particularly for critically ill patients.

As described herein, methods, systems, and devices may safely deliver a gas, such as oxygen, to a patient by diffusing the gas via a medical device (e.g., an intravenous catheter) configured for a high rate of intravenous gas exchange. The medical device may include a permeable membrane that is positioned within a vessel of the patient. The exchange of gas via the permeable membrane may be directly related to the available contact surface area of the membrane and the pressure gradient of the gas inside and outside of the membrane. Given the surface area limitations inside the human central venous system, utilization of higher pressures inside the medical device to achieve higher diffusion rates may advantageously increase the rate of gas exchange via the permeable membrane.

As such, an example system may deliver pressurized gas via the medical device to provide increased rate of diffusivity of the gas to a patient. By controlling the pressure of the gas inside the medical device, and in turn the infusion of gas within the vessel of the patient, the rate of delivery of the gas may be modulated to provide efficacious patient therapy by controlling the pressure of the gas inside the medical device. Such a medical device may enable the delivery of large quantities of oxygen directly to a fluid (e.g., blood) within the vessel and may also be configured to remove other gases from blood, such as carbon dioxide.

In addition, to avoid pressurizing the system, the permeable membrane of the medical device may define nanopores. Gas flowing through the medical device may diffuse through the nanopores to a fluid within the vessel of the patient. As a result of diffusing through the nanopores, the gas may form nanobubbles (e.g., bubbles having diameters of 1 micrometers or less) to the fluid within the vessel of a patient. In this way, the system may deliver large volumes of gas (e.g., oxygen) in the form of nanobubbles and allow for direct mixing with venous blood. Thus, instead of utilizing molecular diffusion to perform gas exchange, the medical device may regulate the production and delivery of gas nanobubbles directly in the bloodstream. Because nanobubbles have relatively large surface areas (e.g., 1 mL of oxygen carried in 100 nanometer bubbles has a surface area of 60 square meters (m²) compared to only 0.06 m² carried in 100 micrometer bubbles) that increase the rate of gas exchange, a smaller medical device configured to deliver nanobubbles may be sufficient to meet a patient's metabolic requirements. As a result, the medical device may be designed with a relatively small form factor, which may be advantageous, e.g., when inserting the medical device into the vessel of a patient.

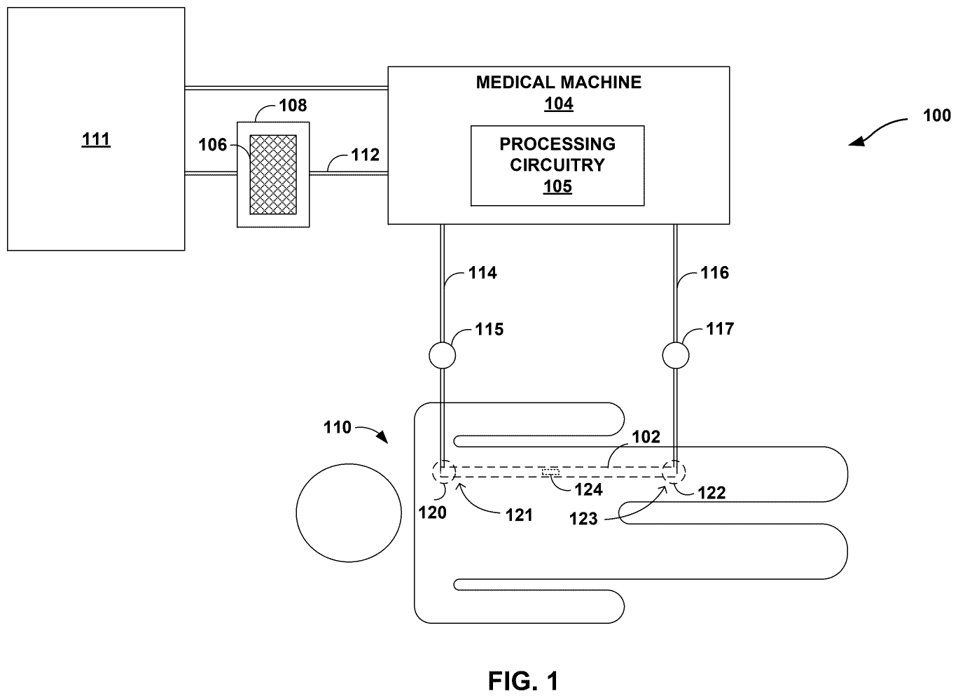

FIG. 1 is a conceptual diagram illustrating an example medical system 100 using an example medical device 102. FIG. 1 illustrates only one particular example of medical system 100, and many other examples of medical system 100 may be used in other instances and may include a subset of the components included in medical system 100 or may include additional components not shown in FIG. 1.

Medical system 100 may include a medical machine 104 configured to provide a pressurized gas 106 (“gas 106”), such as oxygen, nitrous oxide, and/or the like, for a patient therapy (e.g., oxygenation, anesthesia, etc.). For example, medical machine 104 may be configured to provide oxygen via medical device 102 for a patient 110 using oxygen contained in one or more containers, such as a container 108. Container 108 may define a cavity configured to contain gas 106. In some examples, gas 106 may be a mixture of gases (which may or may not include oxygen). In such examples, a percentage by volume of oxygen in gas 106 may be between approximately 0% (e.g., if only the removal of carbon dioxide is desired) and approximately 100%. Gas 106 may be selected for various purposes. For example, gas 106 may be predominately nitrogen if the purpose is to remove a gas, such as carbon dioxide, from the blood of a patient.

Medical machine 104 may regulate the release of gas 106 from container 108 by controlling pressure, temperature, flow rate, and/or the like of gas 106. Medical machine 104 may pressurize container 108 (e.g., using a pressurization device 111, such as a pump, piston, compressor, etc.) containing gas 106 such that a pressure of gas 106 inside medical device 102 exceeds 1 atmospheric pressure (atm). In some examples, medical system 100 may include processing circuitry 105 configured to control medical machine 104 to pressurize container 108. For example, processing circuitry 105 may be configured to control (e.g., by outputting an electrical signal) one or more components configured to pressurize container 108, such as pressurization device 111. Although this disclosure primarily describes processing circuitry 105 of medical machine 104 performing the techniques described herein, processing circuitry of other components of medical system 100 may be configured to perform, at least in part, the techniques of this disclosure.

In some examples, medical machine 104 may be configured to receive gas 106 via a gas line 112 and deliver gas 106 via an infusion line 114. For example, infusion line 114 may provide oxygen to patient 110 via medical device 102. Infusion line 114 may include an inlet flow regulator 115, such as a valve, configured to regulate flow of gas 106 through infusion line 114. For example, inlet flow regulator 115 may at least partially open or close to increase or decrease a transverse cross-sectional area of infusion line 114 through which gas 106 may flow. Medical machine 104 may be configured to receive gas 106 that was not released into patient 110 as well as any waste products (e.g., carbon dioxide) that was removed from patient 110 via a removal line 116. Removal line 116 may include an outlet flow regulator 117, such as a valve, configured to regulate flow of gas 106 (which may now include waste products) through removal line 116. For example, outlet flow regulator 117 may at least partially open or close to increase or decrease a transverse cross-sectional area of removal line 116 through which gas 106 may flow.

Medical system 100 may be configured to intravenously deliver gas 106 to patient 110 (e.g., into one or more of patient's veins) by diffusing gas 106 via medical device 102. Further, medical device 102 may be configured to enable gas 106 to flow through medical device 102 at various pressures, temperatures, flow rates, and/or the like. Medical device 102 may be configured to be intracorporeally positioned within patient 110. In some examples, medical device 102 may be coated with heparin or other anticoagulation medications to reduce the risk of thrombus formation.

Medical device 102 may be in fluid communication with infusion line 114. For example, an infusion port 121 of medical device 102 may be mechanically and fluidically coupled to infusion line 114. Medical device 102 may be in fluid communication with removal line 116. For example, a removal port 123 of medical device 102 may be mechanically and fluidically coupled to removal line 116. A flow channel (not shown), such as an inner lumen, of medical device 102 may extend from infusion port 121 to removal port 123 to form a hydraulic circuit including at least medical machine 104 and medical device 102. Gas 106 may flow through the flow channel of medical device 102. When coupled to infusion port 121, infusion line 114 may extend through an access point 120. When coupled to removal port 123, removal line 116 may extend through an exit point 122.

Medical device 102 may be configured to be intracorporeally positioned within patient 110 (e.g., within a a vessel of patient 110) and diffuse gas 106 into a bloodstream of patient 110. For example, medical device 102 may be inserted via access point 120 (e.g., internal jugular veins, subclavian veins, femoral veins, etc.) on a patient's body until medical device 102 is fully positioned within patient 110 at a desired location (e.g., within a central vein). As shown in FIG. 1A, access point 120 and exit point 122 may not be the same point such that there are distinct percutaneous entrances to patient 110. In some examples, access point 120 and exit point 122 may be the same point such that there is only one percutaneous entrance to patient 110. In such examples, infusion port 121 and removal port 123 may be positioned near each other to allow for a reduced size of the percutaneous entrance through which both infusion line 114 and removal line 116 extend. The permeable membrane may define infusion port 121 and removal port 123.

In accordance with techniques of this disclosure, medical device 102 may include a permeable membrane configured to diffuse gas 106. The permeable membrane may define nanopores extending from an exterior surface of the permeable membrane to the flow channel defined by the permeable membrane. Gas 106 flowing through the flow channel may diffuse through the nanopores to fluid within the vessel of patient 110. The nanopores may be configured (e.g., dimensioned) to cause gas 106 to form nanobubbles in response to diffusion through the nanopores. In some examples, gas 106 forms gas nanobubbles at the exterior surface of the permeable membrane. In some examples, permeable membrane may be formed from a first material configured to be permeable to gas 106 (e.g., dimethyl silicone rubber). In some examples, the permeable membrane may also be non-compliant (i.e., resistant to deformation). Examples of the first material may include carbon, aluminum, one or more polymers, a carbon-based material, a polymer-based material, or any other biocompatible material that allows for nanopores to develop on the surface of the permeable membrane.

Medical device 102 may include a support structure (not shown) configured to control (e.g., resist) expansion of the permeable membrane to reduce the surface tension on the permeable membrane and, thus, the likelihood of the permeable membrane rupturing. For example, the permeable membrane may be positioned within a lumen defined by a non-compliant support structure such that the support structure may mechanically communicate with (e.g., physically contact) the permeable membrane to prevent the permeable membrane from rupturing (e.g., due to expansion) without inhibiting diffusion of gas 106 In some examples, the support structure may be integral with the permeable membrane. Alternatively, the support structure may be a separate structure configured to receive the permeable membrane.

The support structure may be formed from a second material (e.g., polytetrafluoroethylene (PTFE), expanded polytetrafluoroethylene (ePTFE), etc.) that has a significantly higher diffusion rate than the first material such that the support structure does not inhibit diffusion of gas 106, and such that the support structure is non-compliant or at least less compliant than the first material of the permeable membrane. In this way, the permeable membrane may be configured to control the rate of diffusion of gas 106 (e.g., by having a porosity selected to enable a pre-determined rate of gas exchange), and the support structure may be configured to prevent the permeable membrane from rupturing. Example materials for the second material may include carbon, carbon-based materials, a polymer, polymer-based materials, or any other biocompatible material.

The rate of diffusion of gas 106 through medical device 102 may be affected by various factors. For example, the rate of diffusion of gas 106 may be affected by the surface area of the membrane. That is, an increase in surface area of medical device 102 may increase the rate of diffusion of gas 106. In addition, the rate of diffusion of gas 106 may be affected by the pressure within the permeable membrane. For example, an increase in pressure within the permeable membrane of medical device 102 may increase the rate of diffusion of gas 106. Additionally, medical system 100 may be configured to control the flow rate of gas delivery to patient 110. Thus, medical system 100 (and particularly medical device 102) may deliver pressurized gas 106 and at a high flow rate (e.g., a flow rate sufficient to satisfy a patient's metabolic requirements). In some examples, the permeable membrane may be formed (e.g., using three-dimensional (3D) printing) as a shape with an increased surface area (and thus increased rate of diffusion of gas 106).

In some examples, medical device 102 may include a plurality of permeable membranes arranged to increase a surface area of medical device 102 through which gas 106 may diffuse. An example arrangement may define a honeycomb structure (e.g., the permeable membranes of medical device 102 may be parallel to each other and arranged in a hexagonal pattern). Another example arrangement may define a bearing structure having a shape that not only increases a surface area of medical device 102, but also increases the structural integrity (e.g., strength, durability, resilience, etc.) of medical device 102. An example bearing structure may be a lattice structure in which some of the permeable membranes of medical device 102 are parallel to each other, and some of the permeable membranes of medical device 102 are at an angle (e.g., a right angle) to each other. In any case, the plurality of permeable membranes may be in fluid communication with each other. Other arrangements (e.g., an arrangement defining a substantially circular pattern) of the plurality of permeable membranes are possible and contemplated by this disclosure.

The surface area of medical device 102 may also be increased by increasing the length of medical device 102, including the length of the permeable membrane (or membranes) of medical device 102 and the support structure (if one is present) of medical device 102. For example, the length of medical device 102 may extend from a percutaneous entrance near the neck of patient 110 and extend to a leg of patient 110. Medical system 100 may regulate the pressure inside medical device 102 (e.g., based on flow input and gas return resistance) such that the pressure inside medical device 102 is equal to a pre-determined pressure. For example, the permeable membrane may be inflated from a pressure of 1 atm to a pre-determined pressure of 5 atm, increasing the rate of diffusion of gas 106 and thus the amount of gas 106 delivered to patient 110.

Medical device 102 may include a pressure sensor 124 configured to evaluate the pressure inside medical device 102. For example, pressure sensor 124 may be disposed within the permeable membrane of medical device 102. In some examples, processing circuitry 105 of medical machine 104 may control the pressure of gas 106 (e.g., in container 108) in response to signals from pressure sensor 124 to maintain an adequate pressure, and thus adequate diffusion, of gas 106 through the permeable membrane into the bloodstream. In other words, processing circuitry 105 of medical machine 104 and pressure sensor 124 may be components of a closed loop control system configured to control the pressure within medical device 102 based on signals from pressure sensor 124.

For example, processing circuitry 105 of medical machine 104 may be configured to control inlet flow regulator 115 and outlet flow regulator 117 to adjust flow rate of gas 106 into medical device 102 and flow rate of gas 106 out of medical device 102, respectively. For example, responsive to pressure sensor 124 sending a signal to processing circuitry 105 indicating that the pressure within medical device 102 is less than a pre-determined pressure, processing circuitry 105 may control inlet flow regulator 115 to increase the flow rate of gas 106 into medical device 102 and control outlet flow regulator 117 to maintain or decrease the flow rate of gas 106 out of medical device 102. As a result, an amount of gas 106 within medical device 102 (particularly the permeable membrane of medical device 102, which defines a lumen having substantially fixed volume) may increase, increasing the pressure of gas 106 within medical device 102, in turn increasing the rate of diffusion of gas 106 from medical device 102. Thus, medical machine 104 may adjust flow rates of gas 106 into and out of medical device 102 such that the pressure of gas 106 inside medical device 102 is equal to the pre-determined pressure.

In another example, responsive to pressure sensor 124 detecting a sudden change in pressure inside medical device 102 exceeding a threshold value (e.g., a sudden and significant decrease in pressure, which may be associated with a rupture of the permeable membrane), processing circuitry 105 may control pressurization device 111 to stop pressurizing gas 106 and/or immediately stop the flow of gas 106 through the flow channel of medical device 102 (e.g., by controlling inlet flow regulator 115 to stop or substantially stop the flow of gas 106 via infusion line 114).

Medical device 102 may be configured to facilitate gas exchange such that while gas 106 is diffusing from medical device 102, waste molecules, such as carbon dioxide, within patient 110 may diffuse into medical device 102. This may be due to molecular gradients between medical device 102 and the blood that passively cause molecules to move from areas in which the molecules are highly concentrated to areas in which the molecules are not as concentrated. For example, oxygen may diffuse from medical device 102. and carbon dioxide may diffuse into medical device 102.

Medical system 100 may regulate the rate at which the waste molecules are removed by controlling the flow rate of gas 106 through medical device 102. For example, processing circuitry 105 of medical machine 104 may control inlet flow regulator 115 to increase the flow rate of gas 106 into medical device 102 and control outlet flow regulator 117 to increase the flow rate of gas 106 out of medical device 102. As a result, the flow rate of gas 106 through medical device 102 may increase while the pressure of gas 106 remains the same or substantially the same In this way, medical device 102 may increase the rate of removal of molecules 134 from the patient's body. Gas 106 not diffused from medical device 102 and molecules that diffused into medical device 102 may be removed from patient 110 via removal line 116 and flow into medical machine 104 for processing (e.g., recycling of gas 106 and disposal of the waste molecules). It should be understood that medical device 102 may control the rate of delivery of gas 106 to patient's body and removal of the waste molecules from a patient's body independently or at the same time in a manner similar to those described above.

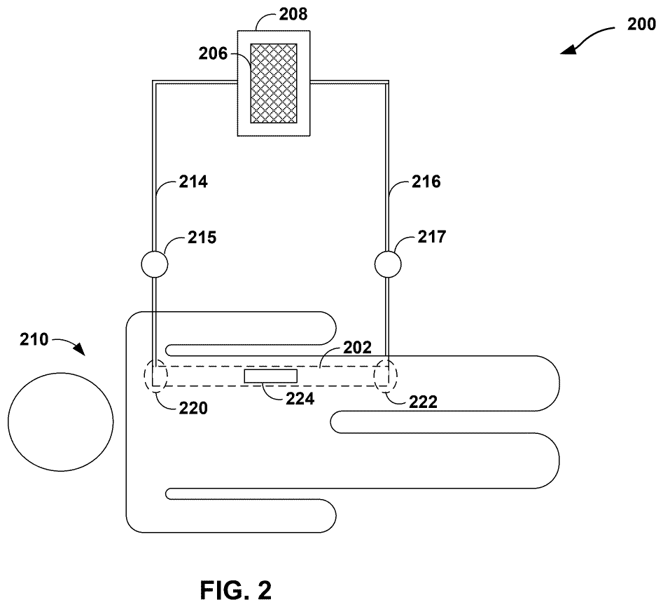

FIG. 2 is a conceptual diagram of a medical system 200 configured to intravenously deliver a gas 206 to a patient, in accordance with one or more aspects of this disclosure. Medical system 200 may be substantially similar to medical system 100 of FIG. 1, except for any differences described herein. As shown in FIG. 2, medical system 200 includes a medical device 202, a container 208, an inlet flow regulator 215, and an outlet flow regulator 217. Inlet flow regulator 215 may control the rate of gas 206 flowing to the patient via an infusion line 214. Outlet flow regulator 217 may be configured to control the rate that gas 206 (which may now include waste products) flows out of the patient via a removal line 216.

As shown in FIG. 2, system 200 does not include a medical machine (e.g., medical machine 104) or a pressurization device (e.g., pressurization device 111). Instead, gas 206 may already be stored at a predetermined pressure (e.g., 5 atm) within container 208. removing the need for system 200 to include a medical machine or pressurization device.

In some examples, medical system 200 may include processing circuitry (e.g., similar to processing circuitry 105) that receives a pressure signal from a pressure sensor 224 and adjusts one or both of inlet flow regulator 215 and outlet flow regulator 217 to maintain a target pressure of gas 206 within medical device 202. In other examples, a user may manually adjust one or both of inlet flow regulator 215 and outlet flow regulator 217 based on pressure signal from pressure sensor 224 (which, e.g., may be displayed to the user via a display device).

Medical system 200 may deliver gas 206 to patient 210 in accordance with techniques of this disclosure (e.g., in a manner similar to that described in FIG. 1). Because medical system 200 does not include as many components as medical system 100, medical system 200 may be more portable than medical system 100. As such, medical system 200 may be more suitable for emergency situations and/or non-hospital settings.

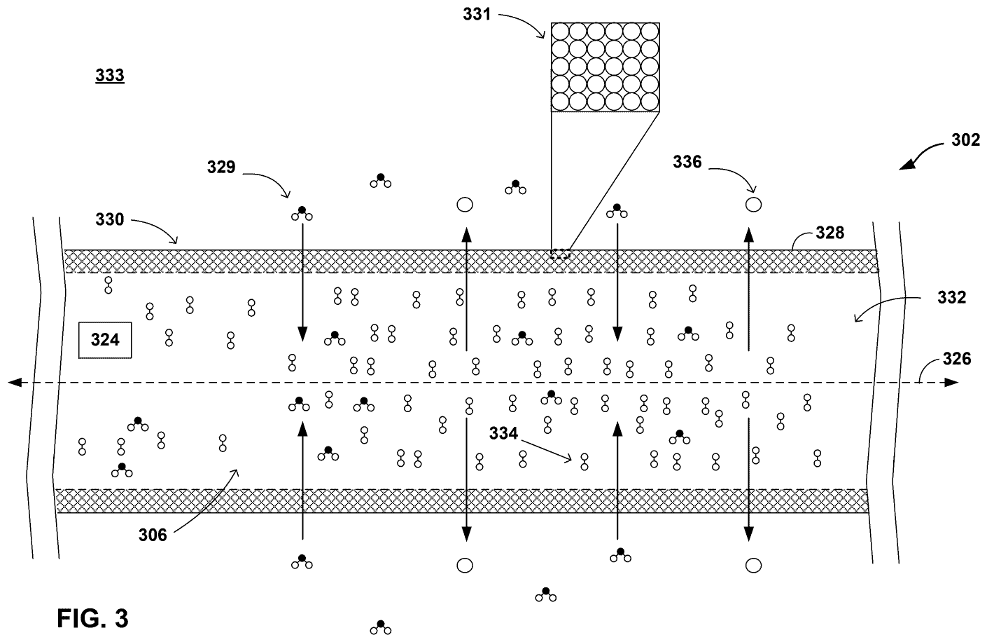

FIG. 3 is a conceptual diagram of a cross-section of an example medical device 302 in accordance with one or more aspects of this disclosure. Medical device 302 may be substantially similar to medical device 102 of FIG. 1 and medical device 202 of FIG. 2, except for any differences described herein. As shown in FIG. 3, a cross-section of medical device 302 is taken parallel to a longitudinal axis 326 of medical device 302. As further shown in FIG. 3, medical device 302 may be in the form of an elongated member that includes a permeable membrane 328. Permeable membrane 328 may define a wall 327 of the elongated member of medical device 302 and a flow channel 332 (illustrated using dashed lines) within wall 327. The elongated member may be in the form of a tube. such as a catheter. The elongated member may be configured to be positioned within a vessel of a patient

Medical device 302 may define an infusion port (e.g., infusion port 121) to receive pressurized gas. Medical device 302 may define a removal port (e.g., removal port 123) to remove unused gas and waste molecules 329 from the patient that originate outside of medical device 302. Permeable membrane 328 may define pores including, but not limited to. nanopores 331 (e.g., pores having a diameter of less than 50 micrometers). Nanopores 331 may extend from an exterior surface 330 of medical device 302 to an interior surface 320 of medical device 302 to provide a path for a gas 306 to reach flow channel 332. A pressure sensor 324 may be positioned within flow channel 332.

Nanopores 331 may be configured to enable diffusion of a gas 306 from out of flow channel 332 and into a fluid 333 (e.g., blood) within the vessel. Nanopores 331 may be configured to form gas nanobubbles 336 (“nanobubbles 336”) at exterior surface 330 of permeable membrane 328 as gas 306 diffuses out from flow channel 332 and into fluid 333 within the vessel. For instance, as shown in zoomed in box 312, pressurized molecules 334 of gas 306 (“gas molecules 334”) may flow out from flow channel 332 and through nanopore 331, form a bubble surface 335 as gas molecules 334 exit nanopores 331, and then coalesce into gas nanobubbles 336 as a result of diffusing through nanopores 331. Thus, medical device 302 may deliver gas molecules 334 as nanobubbles 336 to the bloodstream of a patient, which may increase rate of absorption of gas molecules 334 (e.g., due to increased surface area) while reducing potential health risks (e.g., embolism).

In some examples, each of nanobubbles 336 may have a diameter of 50 micrometers or less. In other examples, each of nanobubbles 336 may have a diameter of 100 micrometers or less. In yet other examples, each of nanobubbles 336 may have a diameter of 250 micrometers or less.

In some examples, permeable membrane 328 may be fabricated from solid, tubular materials configured to define nanopores 331 that allow for the generation of nanobubbles 336 to be released directly in the blood in a controlled, flow-related or pressure-related manner. In some examples, permeable membrane 328 may be formed from a first material configured to be permeable to gas 306 to achieve a desired rate of diffusion of gas 306. For example, permeable membrane 328 may be formed from a material with a high permeability to gas molecules 334 such that the rate of diffusion of gas molecules 334 (at a pre-determined pressure inside medical device 302) through permeable membrane 328 satisfies the metabolic requirements of patient 330. In some examples, the first material may be dimethyl silicone rubber, which is highly permeable to oxygen. However, other materials are contemplated by this disclosure, such as carbon-based materials or polymer-based materials.

Medical device 302 may be configured to facilitate gas exchange. For example, while gas molecules 334 are diffusing from permeable membrane 328, waste molecules 336, such as carbon dioxide, within patient 330 may also diffuse into medical device 302 through permeable membrane 328. This may be due to molecular gradients between medical device 302 and the blood that passively cause molecules 334 to move from areas in which molecules 334 are highly concentrated to areas in which molecules 334 are not as concentrated

Gas molecules 334 (e.g., oxygen molecules) may diffuse from medical device 302. Medical system 300 may regulate the rate at which waste molecules 329 are removed (move from outside of medical device 302 and into flow channel 332) by controlling the flow rate of gas molecules 334 through medical device 302. Gas molecules 334 not diffused from medical device 302 and waste molecules 336 that diffused into medical device 302 may be removed from patient 330 (the locations outside of medical device 302 in FIG. 3) via a removal line (e.g., 116) and flow into a medical machine (e.g., medical machine 104) for processing.

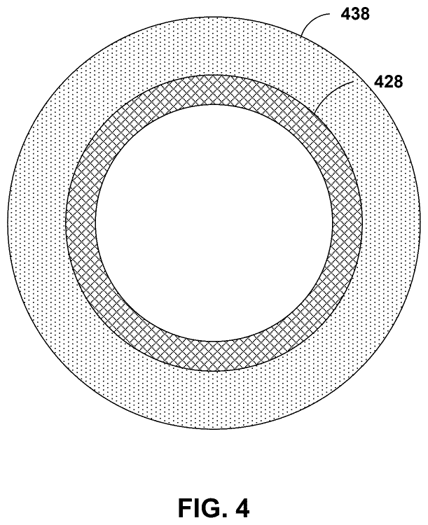

FIG. 4 is a conceptual diagram of a cross-section of a medical device 402, in accordance with one or more aspects of this disclosure. In the example of FIG. 4. the cross-section of medical device 402 is taken perpendicular to a longitudinal axis (not shown) of medical device 402. As shown in FIG. 4, medical device 402 includes a permeable membrane 428 and support structure 438 may be arranged concentrically. In this way, support structure 438 may mechanically communicate with the outer surface of permeable membrane 428 from all radial directions, thereby resisting expansion of permeable membrane 428 that would otherwise occur due to (pressurized) gas molecules flowing through the flow channel defined by permeable membrane 428. In other examples, as shown in FIG. 3, support structure 438 may not be needed as permeable member 428 may, by itself, be configured to maintain the increased pressures of the pressurized gas within medical device 402.

In some examples, support structure 438 may be formed from a material (e.g., PTFE, ePTFE, etc.) different than the material forming permeable membrane 428 that is non-compliant or at least more rigid than permeable membrane 428. That is, permeable membrane 428 may be formed from a first material, and support structure 438 may be formed from a second material. The second material forming support structure 438 may have a significantly higher diffusion rate than the first material such that support structure 438 does not inhibit diffusion of gas molecules. While the cross-sections of permeable membrane 428 and support structure 438 are illustrated in FIG. 4 as being circles, other cross-sectional shapes (e.g., ellipses, squares, rectangles, etc.) are possible and contemplated.

Although primarily described herein as being formed from different materials, permeable membrane 428 and support structure 438 may be formed from the same materials but configured to have different material properties. In general, permeable membrane 428 and support structure 438 may be formed from carbon, a carbon-based material, a polymer, a polymer-based material, or any other biocompatible material. The example materials disclosed herein are not intended to be limiting, and other examples are contemplated by this disclosure.

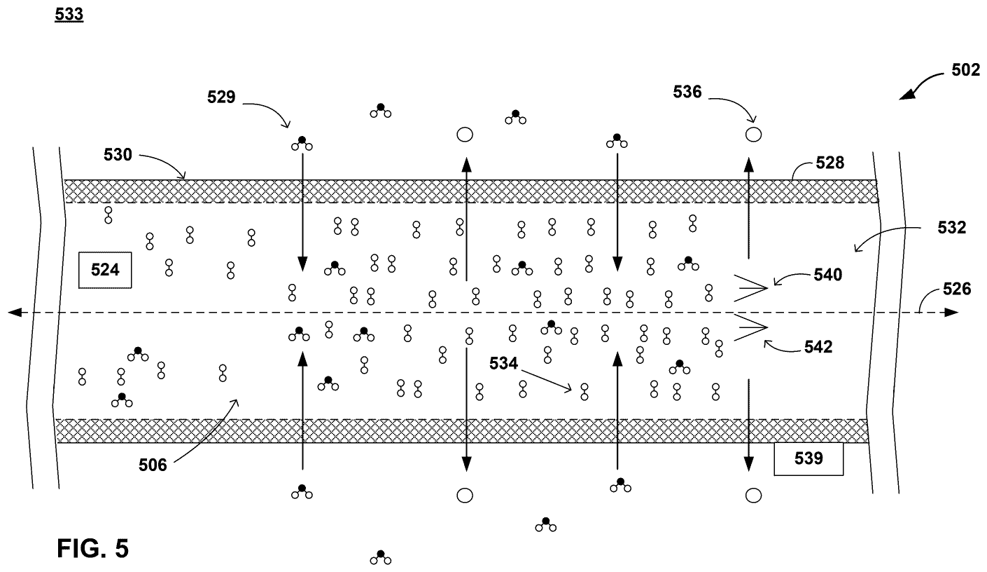

FIG. 5 is a conceptual diagram of a cross-section of an example medical device 502. FIG. 5 illustrates only one particular example of medical device 502 that includes a permeable membrane 528. Many other examples of medical device 502 may be used in other instances and may include a subset of the components included in medical device 502 or may include additional components not shown in FIG. 5 (e.g., a support structure). Medical device 502 may be substantially similar to medical device 102 of FIG. 1. medical device 202 of FIG. 2, medical device 302 of FIG. 3, and/or medical device 402 of FIG. 4, except for any differences described herein. As shown in FIG. 5, a cross-section of medical device 502 is taken parallel to a longitudinal axis 526 of medical device 502. A pressure sensor 524 may be located within a flow channel 532 (illustrated using dashed lines) defined by permeable membrane 528.

Permeable membrane 528 may be configured to enable diffusion of a gas 506 from out of flow channel 532 and into a fluid 533 (e.g., blood) within the vessel. Permeable membrane 528 may be configured to form gas nanobubbles 536 (“nanobubbles 536”) at exterior surface 530 of permeable membrane 528 as gas 506 diffuses out from flow channel 532 and into fluid 533 within the vessel. For instance, as shown in zoomed in box 512, pressurized molecules 534 of gas 506 (“gas molecules 534”) may flow out from flow channel 532 and through nanopore 531, and coalesce into nanobubbles 536 as a result of diffusing through nanopores 531. Thus, medical device 502 may deliver gas molecules 534 as nanobubbles 536 to the bloodstream of a patient, which may increase rate of absorption of gas molecules 534 (e.g., due to increased surface area) while reducing potential health risks (e.g., embolism).

In some examples, medical device 502 includes one or more components configured to agitate gas molecules 534, thereby preventing gas molecules 534 from coalescing into bubbles having a diameter greater than a therapeutic threshold (e.g., bubbles having diameters greater than 50 micrometers). For instance, medical device 502 may use vibration, light, ultrasonic waves, and/or other high-frequency stimuli to perturb gas molecules 534. As an example, medical device 502 may include a vibration generator 539 (e.g., a piezoelectric vibration generator) configured to vibrate medical device 502 and in turn the nanopores of medical device 502. FIG. 5 shows vibration generator 539 positioned on an exterior surface 530 of medical device 502; however, it should be understood that vibration generator 539 may be positioned inside medical device 502, such as on an interior surface 520 of medical device 502. Vibrating medical device 502 may jostle or otherwise perturb gas molecules 534 to prevent gas molecules 534 from coalescing into bubbles having a diameter greater than a therapeutic threshold (e.g., 50 micrometers, 100 micrometers, 250 micrometers, etc.).

In another example, medical device 502 may include a light source 540 configured to emit light into the nanopores of permeable membrane 528. In some examples, light source 540 may include an LED or other light source that transmits light into the nanopores via one or more optical fibers (e.g., a fiber optic cable). Light source 540 may extend through at least a portion of flow channel 532 of medical device 502. The light may impart energy to gas molecules 534 that separate gas molecules 534, in this way preventing coalescence of gas molecules 534 into bubbles having a diameter greater than a therapeutic threshold. In yet another example, medical device 502 may include an ultrasound source 542 configured to emit ultrasonic waves into the nanopores. In some examples, ultrasound source 542 may be an ultrasonic generator or transducer. Ultrasound source 542 may be positioned inside flow channel 532 or outside flow channel 532 of medical device 502. Ultrasound source 542 may deliver ultrasonic waves to the nanopores of medical device 502. Thus, ultrasound source 542 may impart energy to gas molecules 534 that separate gas molecules 534, in this way preventing coalescence of gas molecules 534 into bubbles having a diameter greater than a therapeutic threshold. In some examples, medical device 502 may be coated to modify the hydrophilic properties of the pores of medical device 502 at an exterior surface 530 of medical device 502 (e.g., the interface between the blood of patient 110 and medical device 502).

Medical device 502 may define an infusion port (e.g., infusion port 121) to receive pressurized gas. Medical device 502 may define a removal port (e.g., removal port 123) to remove unused gas and waste molecules 529 from the patient that originate outside of medical device 502.

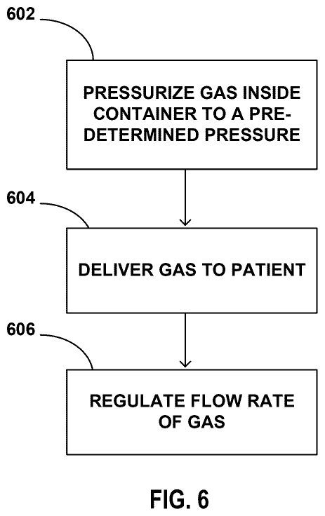

FIG. 6 is a flowchart of an example technique of a system for controlling the pressure of the medical device. Although the example operation of FIG. 6 is described as being performed by medical system 100 of FIG. 1, in other examples some or all of the example operation may be performed by another medical system.

In the example of FIG. 6. medical machine 104 controls pressurization device 111 to pressurize gas 106 inside container 108 (602). In some examples, gas 106 may be a mixture of gases (which may or may not include oxygen). In such examples, a percentage by volume of oxygen in gas 106 may be between approximately 0% and approximately 100%.

In some examples, medical machine 104 may receive gas 106 via a gas line 112 and deliver gas 106 via an infusion line 114. Infusion line 114 may provide oxygen to patient 110 via medical device 102. Infusion line 114 may include inlet flow regulator 115 that regulates flow of gas 106 through infusion line 114. Medical machine 104 may receive gas 106 that was not released into patient 110 as well as waste molecules 136 that were removed from patient 110 via a removal line 116.

Medical system 100 intravenously delivers gas 106 to patient 110 (e.g., into one or more of patient's veins) by diffusing gas 106 via medical device 102 (604). Medical device 102 may enable gas 106 to flow through medical device 102 at various pressures, temperatures, flow rates, and/or the like. Medical device 102 may be intracorporeally positioned within patient 110 and diffuse gas 106 into a bloodstream of patient 110. In some examples, a pre-determined length of medical device 102 may be inserted into patient 110 to ensure that the rate of delivery of gas 106 satisfies the metabolic requirements of patient 110. Medical device 102 may be coated with heparin to reduce the risk of thrombus formation.

Medical device 102 may include a structure configured to diffuse gas 106. For example, medical device 102 may include a permeable membrane (e.g., permeable membrane 328) and a support structure (e.g., support structure 438). The permeable membrane may define nanopores (e.g., nanopores 331) extending from an exterior surface (e.g., exterior surface 330) of the permeable membrane to the flow channel (e.g., flow channel 332) defined by the permeable membrane. Gas 106 flowing through the flow channel may diffuse through the nanopores to the fluid within the vessel of patient 110. The nanopores may be configured (e.g., dimensioned) to cause gas 106 to form nanobubbles in response to diffusion through the nanopores. In some examples, gas 106 forms gas nanobubbles at the exterior surface of the permeable membrane. In some examples, permeable membrane may be formed from a first material configured to be permeable to gas 106 (e.g., dimethyl silicone rubber). In some examples, the permeable membrane may also be non-compliant (i.e., resistant to deformation).

In some examples, medical machine 104 may regulate the release of gas 106 from container 108 by controlling pressure, flow rate, and/or the like of gas 106. For example, responsive to pressure sensor 124 sending a signal to processing circuitry 105 indicating that the pressure within medical device 102 is greater than a pre-determined pressure, processing circuitry 105 may control inlet flow regulator 115 to decrease the flow rate of gas 106 into medical device 102 and control outlet flow regulator 117 to maintain or increase the flow rate of gas 106 out of medical device 102. As a result, an amount of gas 106 within medical device 102 (which defines a substantially fixed volume) may decrease, decreasing the pressure, and thus diffusion, of gas 106 within medical device 102.

In some examples, medical device 102 may facilitate gas exchange. For example, while gas molecules are diffusing from permeable membrane 128, waste molecules, such as carbon dioxide, within patient 110 may diffuse into medical device 102. This may be due to molecular gradients between medical device 102 and the blood that passively cause molecules to move from areas in which the molecules are highly concentrated to areas in which the molecules are not as concentrated.

Medical system 100 may regulate the rate at which waste molecules are removed by controlling the flow rate of gas 106 through medical device 102 (606). For example, processing circuitry 105 of medical machine 104 may control inlet flow regulator 115 to decrease the flow rate of gas 106 into medical device 102 and control outlet flow regulator 117 to decrease the flow rate of gas 106 out of medical device 102. As a result, the flow rate of gas 106 through medical device 102 may decrease while the pressure, and thus diffusion, of gas 106 remains the same or substantially the same. In this way, medical device 102 may decrease the rate of removal of waste molecules from the patient's body.

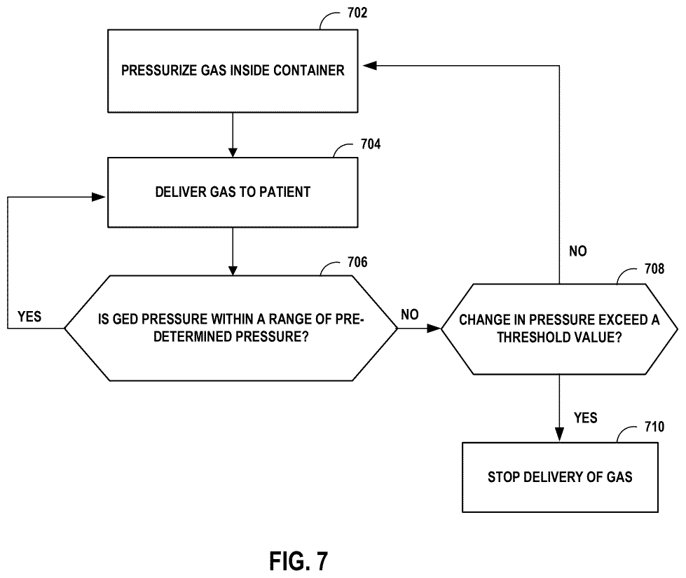

FIG. 7 is a flowchart of an example technique of using a closed loop control system of the system. Although the example operation of FIG. 7 is described as being performed by medical system 100 of FIG. 1, in other examples some or all of the example operation may be performed by another medical system.

In the example of FIG. 7, medical machine 104 of medical system 100 controls pressurization device 111 to pressurize gas 106 inside container 108 to a pre-determined pressure (702). Block 702 of FIG. 7 may be similar, if not substantially similar, to block 602 of FIG. 6. Medical system 100 intravenously delivers gas 106 to patient 110 (e.g., into one or more of patient's veins) by diffusing gas 106 via medical device 102 (704). Block 704 of FIG. 7 may be similar, if not substantially similar, to block 604 of FIG. 6.

Processing circuitry 105 of medical machine 104 determines whether the pressure inside medical device 102 is within a range of the pre-determined pressure of medical device 102 (706). In some examples, processing circuitry 105 of medical machine 104 and pressure sensor 124 may be components of a closed loop control system that controls the pressure within medical device 102 based on a signal from pressure sensor 124. In such examples, if processing circuitry 105 of medical machine 104 determines, based on a measurement from pressure sensor 124, that the pressure inside medical device 102 is less than an upper bound of the range of the pre-determined pressure of medical device 102 and greater than a lower bound of the range of the pre-determined pressure of medical device 102 (“YES” branch of block 706), medical system 100 continues delivering gas 102 to patient via medical device 102 (704).

If processing circuitry 105 of medical machine 104 determines, based on a measurement from pressure sensor 124, that the pressure inside medical device 102 is greater than an upper bound of the range of the pre-determined pressure of medical device 102 or less than a lower bound of the range of the pre-determined pressure of medical device 102 (“NO” branch of block 706), processing circuitry 105 then determines whether the change in pressure exceeds a threshold value (e.g., 10% of the pre-determined pressure, 1 atm, etc.) (708). For example, if pressure sensor 124 measures that pressure inside medical device 102 is 2 atm less than the pre-determined pressure, and if the threshold value is 1 atm. processing circuitry 105 determines that the change in pressure exceeds the threshold value (“YES” branch of block 708). Processing circuitry 105 then controls medical machine 104 to immediately stop the flow of gas 106 through the flow channel of medical device 102 (e.g., by causing inlet flow regulator 115 to completely or substantially completely stop flow of gas 106 via infusion line 114) and/or controls pressurization device 111 to stop pressurizing gas 106 inside container 108 (710).

If processing circuitry 105 of medical machine 104 determines that the change in pressure inside medical device 102 does not exceed a threshold value (“NO” branch of block 708), processing circuitry 105 controls pressurization device 111 to increase or decrease the pressure of gas 106 in container 108 such that the pressure within medical device 102 is within the range of the pre-determined pressure for medical device 102. For example, if pressure sensor 124 measures that pressure inside medical device 102 is 0.02 atm less than the lower bound of the range of the pre-determined pressure for medical device 102, and if the threshold value is 1 atm, processing circuitry 105 controls inlet flow regulator 115 to increase the flow rate of gas 106 into medical device 102 and control outlet flow regulator 117 to maintain or decrease the flow rate of gas 106 out of medical device 102 such that the pressure of gas 106 inside medical device 102 is within the range of the pre-determined pressure (e.g., equal to the lower bound of the range of the pre-determined pressure).

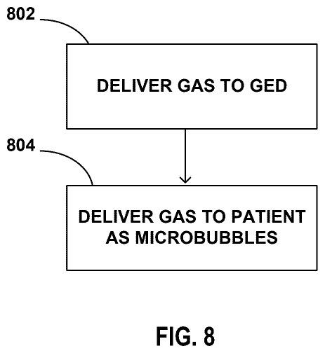

FIG. 8 is a flowchart of an example technique of using a closed loop control system of the system. Although the example operation of FIG. 8 is described as being performed by medical system 500 of FIG. 5, in other examples some or all of the example operation may be performed by another medical system.

In the example of FIG. 7, gas molecules 534 are delivered to medical device 502 (802). Gas molecules 534 may flow through flow channel 532 defined by permeable membrane 528. Medical device 502 may deliver gas molecules 534 to a patient in the form of nanobubbles (804). In some examples, medical device 502 includes one or more components configured to agitate gas molecules 534 to prevent gas molecules 534 from coalescing into bubbles having a diameter greater than a therapeutic threshold. For instance, medical device 502 may use vibration, light, ultrasonic waves, and/or other high-frequency stimuli to perturb gas molecules 534. As an example, vibration generator 539 positioned on an exterior surface of medical device 502 or within medical device 502 may vibrate medical device 502. Vibrating medical device 502 may jostle or otherwise perturb gas molecules 534 to prevent coalescence of gas molecules 534 into bubbles having a diameter greater than a therapeutic threshold.

In another example, medical device 502 may include a light source 540 configured to emit light within flow channel 532. In some examples, light source 540 may transmit light via one or more optical fibers extending through at least a portion of flow channel 532 of medical device 502. The light may impart energy to gas molecules 534 that separate gas molecules 534, in this way preventing coalescence of gas molecules 534 into bubbles having a diameter greater than a therapeutic threshold. In yet another example, medical device 502 may include an ultrasound source 542 configured to emit ultrasonic waves through flow channel 532. In some examples, ultrasound source 440 may be an ultrasonic generator or transducer. Ultrasound source 542 may be positioned inside flow channel 532 or outside flow channel 532 of medical device 502. Ultrasound source 542 may deliver ultrasonic waves that impart energy to gas molecules 534 that separate gas molecules 534, in this way preventing coalescence of gas molecules 534 into bubbles having a diameter greater than a therapeutic threshold. In some examples, medical device 502 may be coated to modify the hydrophilic properties of the pores of medical device 502 at the exterior surface of medical device 502.

The following examples are illustrative of the techniques described herein.

Example 1: A medical device includes an elongated member configured to be inserted into a vessel of a patient, wherein the elongated member includes a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel. and wherein the nanopores are configured to form gas nanobubbles at the exterior surface of the permeable membrane as the pressurized gas diffuses out from the flow channel and into the fluid within the vessel.

Example 2: The medical device of example 1, wherein the elongated member is configured to allow the pressurized gas to flow through the flow channel at a high flow rate, and wherein the elongated member is configured to maintain the pressurized gas at a higher pressure than a pressure of the vessel.

Example 3: The medical device of example 1 or 2, wherein each nanobubble of the gas nanobubbles has a diameter of 50 micrometers or less.

Example 4: The medical device of any of examples 1 to 3, further including a support structure surrounding the permeable membrane, wherein the support structure is configured to resist expansion of the permeable membrane under pressure of the pressurized gas.

Example 5: The medical device of any of examples 1 to 4, further including a light source configured to emit light into the nanopores, wherein the light is configured to prevent the gas from coalescing into bubbles having a diameter greater than a therapeutic threshold.

Example 6: The medical device of example 5, wherein the light source is configured to transmit light via one or more optical fibers.

Example 7: The medical device of any of examples 1 to 6, further including a vibration generator configured to vibrate the nanopores to prevent the pressurized gas from coalescing into bubbles having a diameter greater than a therapeutic threshold.

Example 8: The medical device of example 7, wherein the vibration generator includes a piezoelectric vibration generator.

Example 9: The medical device of any of examples 1 to 8, further including an ultrasound source configured to emit ultrasonic waves into the nanopores to prevent the pressurized gas from coalescing into bubbles having a diameter greater than a therapeutic threshold.

Example 10: The medical device of any of examples 1 to 9, wherein the elongated member is configured to contain the pressurized gas at a predetermined pressure.

Example 11: The medical device of example 10, wherein the predetermined pressure is equal to or greater than 5 atmospheric pressure.

Example 12: The medical device of any of examples 1 to 11, wherein the pressurized gas includes oxygen.

Example 13: A method includes inserting a medical device into a vessel of a patient, wherein the medical device includes an elongated member including a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel; and delivering the pressurized gas through the flow channel of the permeable membrane.

Example 14: The method of example 13, wherein delivering the gas includes delivering the pressurized gas at a high flow rate, and wherein the elongated member is configured to maintain the pressurized gas at a higher pressure than a pressure of the vessel.

Example 15: The method of example 13 or 14, wherein each of the gas nanobubbles has a diameter of 50 micrometers or less.

Example 16: The method of any of examples 13 to 15, wherein a support structure surrounds the permeable membrane, and wherein the support structure is configured to resist expansion of the permeable membrane under pressure of the pressurized gas.

Example 17: The method of any of examples 13 to 16, further including emitting, by a light source of the medical device, light into the nanopores to prevent the gas from coalescing into bubbles having a diameter greater than a therapeutic threshold.

Example 18: The method of example 17, wherein the light source is configured to transmit light via one or more optical fibers.

Example 19: The method of any of examples 13 to 18, further including vibrating. by a vibration generator of the medical device, the nanopores to prevent the gas from coalescing into bubbles having a diameter greater than a therapeutic threshold.

Example 20: The method of example 19, wherein the vibration generator includes a psiezoelectric vibration generator.

Example 21: The method of any of examples 13 to 20, further including emitting, by an ultrasound source of the medical device, emit ultrasonic waves into the nanopores to prevent the gas from coalescing into bubbles having a diameter greater than a therapeutic threshold.

Example 22: The method of any of examples 13 to 21, wherein the pressurized gas is pressurized to a predetermined pressure.

Example 23: The method of any of examples 13 to 22, wherein the predetermined pressure is equal to or greater than 5 atmospheric pressure.

Example 24: The method of any of examples 13 to 23, wherein the pressurized gas includes oxygen.

Example 25: A system includes a container defining a cavity configured to contain a pressurized gas; and a medical device, fluidically coupled to the container includes an elongated member configured to be inserted into a vessel of a patient, wherein the elongated member includes a permeable membrane that defines a wall of the elongated member and a flow channel within the wall, wherein the permeable membrane defines nanopores extending from an exterior surface of the permeable membrane to the flow channel, wherein the nanopores are configured to enable diffusion of the pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

Example 26: The system of example 25, further including an inlet flow regulator configured to control a flow rate of the gas into the medical device from the container.

Example 27: The system of example 25 or 26, further including an outlet flow regulator configured to control a flow rate of the gas out of the medical device.

Example 28: The system of any of examples 25 to 27, further including a pressure sensor configured to sense a pressure within the medical device.

Example 29: A system includes a container defining a cavity configured to contain a pressurized gas; a pressurization device configured to pressurize gas; and a medical device, fluidically coupled to the container includes an elongated member configured to be inserted into a vessel of a patient, wherein the elongated member includes a permeable membrane that defines a wall of the elongated member and a flow channel within the wall, wherein the permeable membrane defines nanopores extending from an exterior surface of the permeable membrane to the flow channel, wherein the nanopores are configured to enable diffusion of the pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

Example 30: The system of example 29, further including a pressure sensor configured to sense a pressure within the medical device.

Example 31: The system of example 29 or 30, further including a closed loop control system configured to control the pressure within the medical device based on a signal from the pressure sensor.

Example 32: The system of example 31, wherein the system further includes: an inlet flow regulator configured to control a flow rate of the gas into the medical device; and an outlet flow regulator configured to control a flow rate of the gas out of the medical device, and wherein the closed loop control system is configured to control the pressure within the medical device by controlling the inlet flow regulator and the outlet flow regulator.

The techniques described in this disclosure may be implemented, at least in part, in hardware, software, firmware, or any combination thereof. For example, various aspects of the techniques may be implemented within one or more microprocessors. DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, FRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

Various examples have been described. These and other examples are within the scope of the following claims.

1. A medical device comprising:
an elongated member configured to be inserted into a vessel of a patient, wherein the elongated member comprises a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.

2. The medical device of claim 1, wherein the elongated member is configured to allow the pressurized gas to flow through the flow channel at a high flow rate, and wherein the elongated member is configured to maintain the pressurized gas at a higher pressure than a pressure of the vessel.

3. The medical device of claim 1, wherein each nanobubble of the gas nanobubbles has a diameter of 50 micrometers or less.

4. The medical device of claim 1, further comprising a support structure surrounding the permeable membrane, wherein the support structure is configured to resist expansion of the permeable membrane under pressure of the pressurized gas.

5. The medical device of claim 1, further comprising a light source configured to emit light into the nanopores, wherein the light is configured to prevent the gas from coalescing into bubbles having a diameter greater than a therapeutic threshold.

6. The medical device of claim 5, wherein the light source is configured to transmit light via one or more optical fibers.

7. The medical device of claim 1, further comprising a vibration generator configured to vibrate the nanopores to prevent the pressurized gas from coalescing into bubbles having a diameter greater than a therapeutic threshold.

8. The medical device of claim 7, wherein the vibration generator comprises a piezoelectric vibration generator.

9. The medical device of claim 1, further comprising an ultrasound source configured to emit ultrasonic waves into the nanopores to prevent the pressurized gas from coalescing into bubbles having a diameter greater than a therapeutic threshold.

10. The medical device of claim 1, wherein the elongated member is configured to contain the pressurized gas at a predetermined pressure.

11. The medical device of claim 10, wherein the predetermined pressure is equal to or greater than 5 atmospheric pressure.

12. The medical device of claim 1, wherein the pressurized gas comprises oxygen.

13. The medical device of claim 1, wherein the medical device is fluidically coupled to a container defining a cavity configured to contain a pressurized gas.

14. The medical device of claim 13, wherein at least one of the medical device or the container comprises an inlet flow regulator configured to control a flow rate of the gas into the medical device from the container.

15. The medical device of claim 13, wherein at least one of the medical device or the container comprises an outlet flow regulator configured to control a flow rate of the gas out of the medical device.

16. A method comprising:
inserting a medical device into a vessel of a patient, wherein the medical device includes an elongated member including a permeable membrane that defines a wall of the elongated member and a flow channel within the wall, wherein the permeable membrane defines nanopores extending from an exterior surface of the permeable membrane to the flow channel, wherein the nanopores are configured to enable diffusion of a pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel; and
delivering the pressurized gas through the flow channel of the permeable membrane.

17. The method of claim 16, wherein delivering the gas comprises delivering the pressurized gas at a high flow rate, and wherein the elongated member is configured to maintain the pressurized gas at a higher pressure than a pressure of the vessel.

18. The method of claim 16, further comprising emitting, by a light source of the medical device and via one or more optical fibers, light into the nanopores to prevent the gas from coalescing into bubbles having a diameter greater than a therapeutic threshold.

19. The method of claim 16, further comprising vibrating, by a vibration generator of the medical device, the nanopores to prevent the gas from coalescing into bubbles having a diameter greater than a therapeutic threshold.

20. A system comprising:
a container defining a cavity configured to contain a pressurized gas; and
a medical device, fluidically coupled to the container, comprising an elongated member configured to be inserted into a vessel of a patient, wherein the elongated member includes a permeable membrane that defines a wall of the elongated member and a flow channel within the wall, wherein the permeable membrane defines nanopores extending from an exterior surface of the permeable membrane to the flow channel, wherein the nanopores are configured to enable diffusion of the pressurized gas from out of the flow channel and into a fluid within the vessel, and wherein the nanopores are configured to form gas nanobubbles at the exterior surface of the permeable membrane as the pressurized gas diffuses out from the flow channel and into the fluid within the vessel.
